(19) United States
(12) Patent Application Publication
(10) Pub. No.: US 2022/0280221 A1
(43) Pub. Date: Sep. 8, 2022

(54) SYSTEMS, APPARATUSES, AND METHODS FOR VAPOR GENERATION

(71) Applicant: Boston Scientific Scimed, Inc., Maple Grove, MN (US)

(72) Inventors: Steven T. CARLSON, St. Paul, MN (US); Niraj Prasad RAUNIYAR, Plymouth, MN (US); Mark David MIRIGIAN, Piltown, (IE)

(73) Assignee: Boston Scientific Scimed, Inc., Maple Grove, MN (US)

(21) Appl. No.: 17/652,715

(22) Filed: Feb. 28, 2022

Related U.S. Application Data
(60) Provisional application No. 63/156,079, filed on Mar. 3, 2021.

(51) Int. Cl. A61B 18/08

(52) U.S. Cl. CPC A61B 18/082; A61B 2018/00577

(57) ABSTRACT

In an exemplary arrangement, a vapor generator for use in a medical device may include a heating core in fluid communication with a source of fluid and defining at least one fluid pathway along which fluid from the source of fluid travels. The at least one fluid pathway may include one or more surfaces that generate turbulence in the fluid. The vapor generator may also include a coil disposed about the heating core, wherein the coil is configured to receive a current so as to heat the fluid traveling along the at least one fluid pathway, thereby generating a vapor.

### CROSS-REFERENCE TO RELATED APPLICATIONS

This application claims the benefit of priority from U.S. Provisional Application No. 63/156,079, filed on Mar. 3, 2021, which is incorporated by reference herein in its entirety.

### TECHNICAL FIELD

The present disclosure relates generally to devices, systems, and methods for vapor generation and, in particular, vapor generators for vapor ablation devices.

### BACKGROUND

Certain medical conditions, such as conditions of the prostate, may be treated by ablation, including by vapor ablation. Such ablation may be performed using a device having a sheath that is inserted into a body lumen or otherwise into a body of a patient. Vapor (e.g., water vapor) may be released from the device in order to ablate tissue, such as prostate tissue, or otherwise treat tissue. The vapor may be generated by a vapor generator of the device. It may be desirable for the generator to occupy a smaller footprint than current generators and to efficiently produce high-quality vapor.

The systems, devices, and methods of the current disclosure may rectify some of the deficiencies described above, and/or address other aspects of the prior art.

### SUMMARY

In an exemplary arrangement, a vapor generator for use in a medical device may include a heating core in fluid communication with a source of fluid and defining at least one fluid pathway along which fluid from the source of fluid travels. The at least one fluid pathway may include one or more surfaces that generate turbulence in the fluid. The vapor generator may also include a coil disposed about the heating core, wherein the coil is configured to receive a current so as to heat the fluid traveling along the at least one fluid pathway, thereby generating a vapor.

The coil may be configured to inductively heat the fluid.

The heating core may include a latticed body, and one or more struts of the latticed body may define the one or more surfaces that generate the turbulence in the fluid.

The heating core may further include a sheath disposed about the latticed body.

The latticed body may include Inconel.

The latticed body may have an approximately cylindrical shape.

The latticed body may define a plurality of openings defined by a plurality of struts.

All of the openings may be in fluid communication with one another.

The at least one fluid pathway may define a plurality of routes of fluid travel along (a) a path approximately parallel to a longitudinal axis of the latticed body and (b) a path transverse to the longitudinal axis of the latticed body.

At least a portion of the heating core may be disposed within a needle of the medical device.

A portion of the needle having the heating core may be disposed within a shaft insertable into a body lumen of a subject.

The heating core may include a tube, and the tube may define a lumen having a textured wall surface. The textured wall surface may generate the turbulence in the fluid.

The tube may form a coil.

The heating core may include two or more coils.

The tube may have a non-circular cross-section.

In another exemplary arrangement, a vapor generator for use in a medical device may include a latticed body in fluid communication with a source of fluid and defining a plurality of fluid pathways along which fluid from the source of fluid travels. The latticed body may include a plurality of struts defining a plurality of openings. The vapor generator may further include a coil disposed about the latticed body. The coil may be configured to receive a current so as to heat the fluid traveling along the plurality of fluid pathways, thereby generating a vapor.

The plurality of fluid pathways define routes of fluid travel along (a) a path approximately parallel to a longitudinal axis of the latticed body and (b) a path transverse to the longitudinal axis of the latticed body.

At least a portion of the latticed body may be disposed within a needle of the medical device.

The latticed body may have an approximately cylindrical shape.

In a further exemplary arrangement, a vapor generator for use in a medical device may include a latticed body in fluid communication with a source of fluid and defining a plurality of fluid pathway along which fluid from the source of fluid travels. The plurality of fluid pathways may define routes of fluid travel along (a) a path approximately parallel to a longitudinal axis of the latticed body and (b) a path transverse to the longitudinal axis of the latticed body. The vapor generator may further include a coil disposed about the latticed body. The coil may be configured to receive a current so as to heat the fluid traveling along the plurality of fluid pathways, thereby generating a vapor.

It may be understood that both the foregoing general description and the following detailed description are exemplary and explanatory only and are not restrictive of the invention, as claimed. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” As used herein, the term “proximal” means a direction closer to an operator and the term “distal” means a direction further from an operator. Although vapor ablation is referenced herein, such references should not be construed as limiting. The examples disclosed herein may also be used with other types of ablation mechanisms (e.g., cryoablation, RF ablation, or other types of ablation) or with other devices not relating to ablation.

### BRIEF DESCRIPTION OF THE DRAWINGS

The accompanying drawings, which are incorporated in and constitute a part of this specification, illustrate examples of the present disclosure and together with the description, serve to explain the principles of the disclosure.

FIG. 1 is a side view of a portion of an exemplary ablation device.

FIG. 2 depicts an exemplary vapor generator for use with the ablation device of FIG. 1.

FIGS. 3A-5C depict exemplary heating cores for use with the vapor generator of FIG. 2.

FIG. 6 depicts an exemplary shaft and distal tip of an ablation device, such as the ablation device of FIG. 1.

FIGS. 7A-10C depict exemplary heating cores for use with the shaft and distal tip of FIG. 6.

FIG. 11 depicts an exemplary vapor generator for use with an ablation device, such as the ablation device of FIG. 1.

FIGS. 12A-12D depict exemplary tubing for use with, for example, fluid coils.

FIGS. 13 and 14 depict exemplary fluid coils.

### DETAILED DESCRIPTION

A vapor generator for a vapor ablation device may include a radio frequency (“RF”) coil surrounding a conductive body that defines a pathway through which fluid (e.g., water) may pass. The conductive body may be formed via additive manufacturing, such as three-dimensional (“3D”) metal printing. A current through the RF coil may produce an electromagnetic flux, causing current(s) (e.g., eddy currents) in the conductive body, and heating of the conductive body. The heating of the conductive body may cause heating of water passing therethrough, thereby generating water vapor. The conductive body may include, for example, a conductive mesh, lattice, or one or more coils. The generator (RF coil and conductive body) may be disposed within a handle of the vapor ablation device or within a shaft of the vapor ablation device. The water vapor may be conveyed to a treatment site in order to therapeutically treat a tissue. For example, the vapor may ablate the tissue. In one example, the ablated tissue may be prostate tissue, and the ablation may treat benign prostatic hyperplasia (BPH).

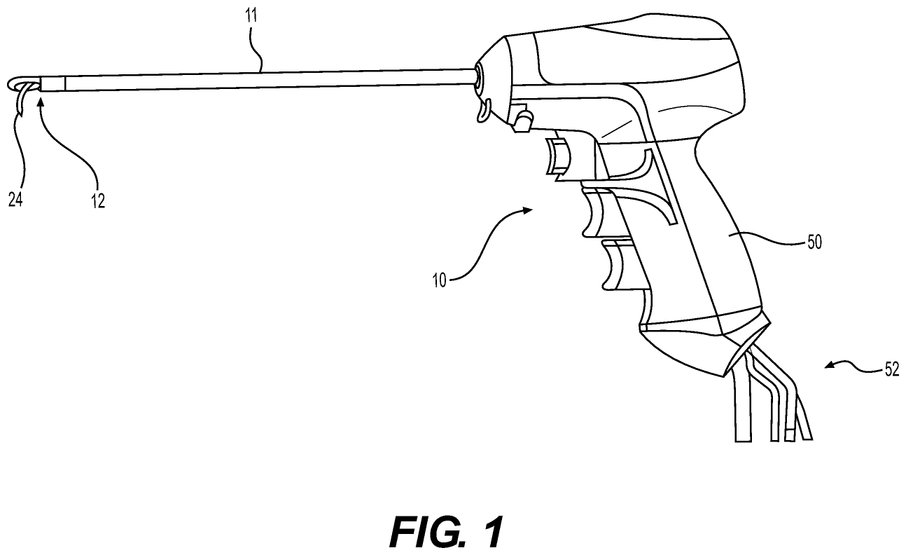

FIG. 1 shows a side view of an exemplary distal assembly of an ablation device 10. Ablation device 10 may include a handle 50 and a shaft 11. Shaft 11 may be insertable into a body lumen of a patient or otherwise into a body of a patient (e.g., through a tissue of a patient, such as via a transperineal route). Shaft 11 may have a distal tip 12. Handle 50 may be configured to be gripped by a user.

A needle 24 may be extendable and/or retractable from distal tip 12. Needle 24 may be a member having a central lumen or channel extending from a proximal end of needle 24 toward a distal tip of needle 24, and a plurality of apertures near the distal tip of needle 24. The plurality of apertures may be configured to communicate the contents of the central lumen or channel (e.g., vapor, steam) to surrounding tissue into which needle 24 is positioned, received, or otherwise inserted. For example, the central lumen or channel of needle 24 may be configured to receive vapor therein (e.g., via a vapor generator) and to deliver the vapor to tissue via the apertures. Needle 24 may be configured to have a first, insertion configuration, in which needle 24 is contained, received, or otherwise positioned within shaft 11 (e.g., such that no portion of needle 24 extends radially outwardly of distal tip 12, relative to a longitudinal axis of distal tip 12). Needle 24 may have a second, treatment configuration (FIG. 1), in which needle 24 is extended out of distal tip 12 (e.g., distally past and/or radially outwardly of distal tip 12, relative to the longitudinal axis of distal tip 12). In the treatment configuration, needle 24 may curve radially outward relative to the longitudinal axis of shaft 11.

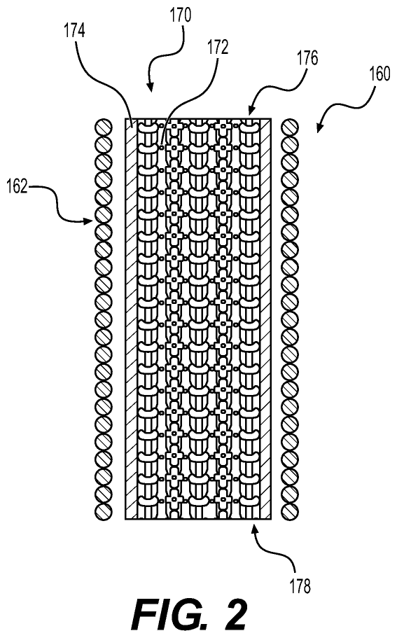

Handle 50 may include cabling 52 extending proximally from a proximal end of handle 50. Cabling 52 may transmit power, fluids, signals, etc. to handle 50 or other portions of ablation device 10 (e.g., shaft 11). In an example, cabling 52 may transmit fluid, such as water, from a fluid source to ablation device 10. In some embodiments, a vapor generator (to be discussed in further detail, with respect to FIGS. 2-14, herein) may be disposed within ablation device 10 (e.g., in handle 50 or shaft 11). Fluid to be passed through the vapor generator may be housed within ablation device 10 or may be transmitted to ablation device 10 via cabling 52. In other embodiments, the vapor generator may be disposed externally to ablation device 10, and vapor may be transmitted from the vapor generator to ablation device 10 via cabling 10.

FIG. 2 shows an exemplary vapor generator 160. Vapor generator 160 may be used to deliver vapor to a device, such as ablation device 10. In examples, vapor generator 160 may be disposed within handle 50 or within shaft 11. In other examples, vapor generator 160 may be disposed externally of ablation device 10, as described above. Vapor generator 160 may include a coil 162 and a heating core 170. A current through coil 162 may serve to heat fluid traveling through core 170, as described in further detail below. Vapor generator 160 may serve to inductively heat the fluid.

Coil 162 may carry an RF current or other alternating current. Coil 162 may have any features that assist coil 162 in carrying current. Coil 162 may be made from any suitable material and may contain any suitable number of windings. For example, coil 162 may include a Litz wire (a type of wire that is particularly efficient at carrying RF energy). As alternating current passes through coil 162, the current may generate one or more magnetic fields. Coil 162 may be constructed from turned wire. Alternatively, coil 162 may be formed by additive manufacturing methods, including, for example, extrusion, binder jetting, powder bed fusion, or any other suitable form of additive manufacturing/3D metal printing. Coil 162 may include an insulating material to prevent current from coil 162 from passing to other structures or between turns of coil 162.

Core 170 may include a latticed body 172 and a sheath 174. Latticed body 172 may have an approximately cylindrical overall shape having a first end 176 and a second end 178. Latticed body may include a plurality of cells having a plurality of struts meeting at a plurality of nodes. Latticed body 172 may include one cell structure that is repeated a plurality of times in a pattern or a plurality of cell structures that may be repeated in a pattern. Each cell may include an opening defined by the struts. The openings of the plurality of cells may all be in fluid communication with one another. Fluid may flow from first end 176 to second end 178, via the fluidly connected openings. Alternatively, the openings may define a plurality of pathways that extend from first end 176 to second end 178 but are not in fluid communication with one another between first end 176 and second end 178. Latticed body 172 may be formed of one of more conductive materials and may be formed via any of the additive manufacturing techniques described above. For example, latticed body 172 may be formed of a printable material exhibiting high thermal conductivity such as copper. Additionally or alternatively, latticed body 172 may be formed of a printable material exhibiting a combination of high thermal conductivity and biocompatibility such as 17-4 stainless, Inconel, 316L stainless, Cobalt Chromium (CoCr), etc. A material of latticed body 172 may be uniform or may be varied in order to maximize efficiency of the energy transfer described below. In some arrangements, a material of latticed body 172 may be formed via powder bed fusion.

Sheath 174 may encase an outer surface of latticed body 172 between first end 176 and second end 178, such that fluid may not pass out of the side of latticed body 172 between first end 176 and second end 178. Sheath 174 may be formed of any suitable material, such as metal or non-metal. Sheath 174 may be formed by any suitable manufacturing method, such as from a sheet of material or via additive manufacturing. Sheath 174 may be formed of the same material as latticed body 172 or from a different material. Sheath 174 may be fixed to latticed body 172 via, for example, adhesive, welding, or by frictional fit. Together, sheath 174 and latticed body 172 may form a tube (sheath 174) having a lattice infill (latticed body 172).

Core 170 may be positioned in a center of coil 162. As shown in FIG. 2, core 170 may be separated from coil 162 by a gap. Alternatively, coil 162 may abut core 170 but may be insulated so that current does not flow from coil 162 to core 170 and the insulation may have properties to withstand heating of core 170. As a current (e.g., RF current) passes through coil 162, coil 162 may generate one or more magnetic fields. The magnetic field(s) may induce eddy currents within the conductive material of latticed body 172. The eddy currents flowing through the resistance of the conductive material may generate heat within the latticed body 172. The plurality of struts of latticed body 172 may be configured to provide a desired heating pattern. For example, more heat may be generated in some portions of latticed body 172 than in other portions of latticed body. In an example, more heat may be generated in radially outer portions of latticed body 172 than in radially inner portions of latticed body 172 (relative to a longitudinal axis of core 170). Greater amounts of heat may be generated in portions of latticed body where additional heat is required in order to generate high quality, consistent vapor throughout an entirety of latticed body 172. Materials of latticed body 172 may be chosen to facilitate a desired heating pattern. Alternatively, the plurality of struts of latticed body 172 may be configured so as to heat in an even matter. For example, a thickness of the struts may be chosen to facilitate even heating. Eddy currents may also be generated in sheath 174, thereby heating sheath 174.

As current flows through coil 162, a source of fluid (e.g., water) may cause fluid to flow into first end 176 of latticed body 172. Latticed body 172 may define a fluid pathway through which the fluid from the source of fluid may travel. The fluid pathway may include numerous branches, and a particular route the fluid follows may vary. For example, routes may facilitate movement of fluid approximately parallel to a longitudinal axis of latticed body 172 and transverse to the longitudinal axis of latticed body 172. As fluid passes the struts of the latticed body 172 and/or sheath 174, the heat may be transferred from latticed body 172 and/or sheath 174 to the fluid, thereby heating the fluid. Flow through latticed body 172 may be turbulent and provide a large amount of surface area to contact the fluid and heat it. The turbulence may promote an efficient heat flow. The fluid may be sufficiently heated such that a vapor is generated. For example, substantially all of the fluid may be transformed to vapor. The configuration of latticed body 172 may provide a large amount of surface area for the fluid to contact, providing efficient heating. As compared with a coil through which fluid flows through in order to be heated, latticed body 172 may provide the same heating (due to the large surface area of latticed body 172 that contacts the fluid) while occupying a smaller footprint. The smaller footprint may facilitate manufacturing efficiencies and/or a smaller size of ablation device 10. For example, vapor generator 160 may occupy a smaller amount of space in handle 50 of ablation device 10, allowing handle 50 to be manufactured with a smaller size, making room for additional components (e.g., control boards or other electronics), allowing alternative positions of vapor generator 160 within handle 50, or easing manufacturing of handle 50 due to a greater availability of space. Furthermore, as discussed above, a smaller profile of vapor generator 160 may allow placement of generator 160 in shaft 11.

As compared to a coil, for example, a length of a path of the fluid may be shorter through latticed body 172, providing for faster travel through vapor generator 160, thereby resulting in more efficient heat transfer. That is, an overall length of the path traveled by a fluid passing through the lattice may be shorter than an overall length of the path of fluid passing around the various turns of a coil. The efficient heat transfer from core 170 to the fluid produces a high-quality, uniform vapor. Vapor quality is the ratio of water to vapor in a mixture. For example, a mixture may start with 100% water and 0% vapor. However, as heat is added, a phase change occurs and the percentage amount of vapor increases. Eventually, all water is converted to vapor such the mixture includes 0% water and 100% vapor. Higher vapor quality (e.g., the higher the ratio) is ideal for treating tissue (e.g., prostate tissue) as vapor is a gas and may pass through interstitial space in the tissue easier (e.g., more freely) than a liquid.

Vapor generator 160 may have additional elements that are not shown in FIG. 2. For example, a thermocouple may be coupled to a portion of core 170, such as latticed body 172, in order to allow for control of heating of core 170.

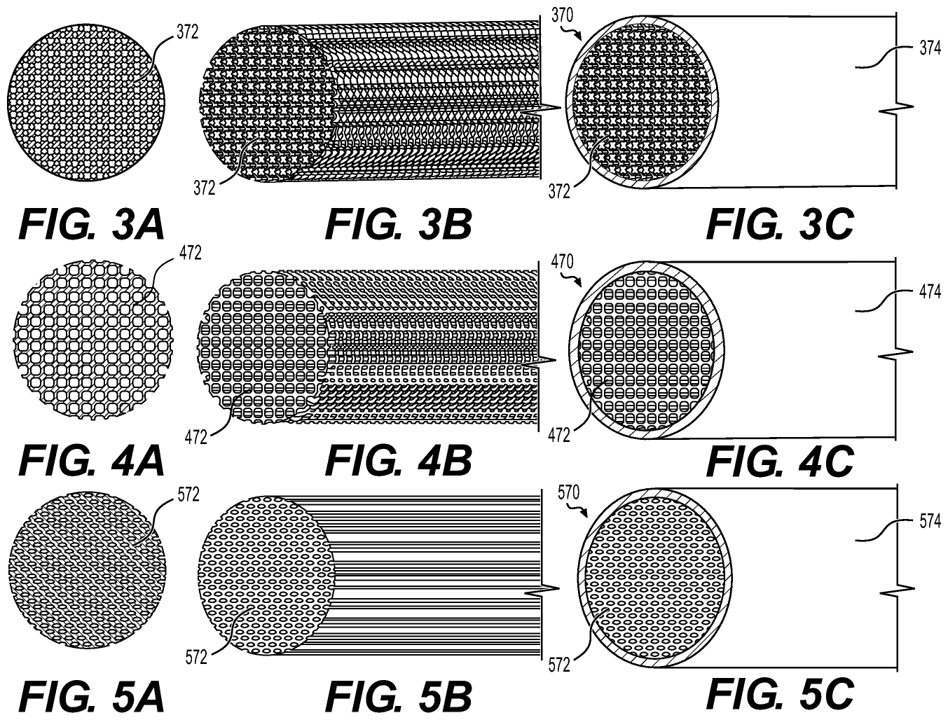

FIGS. 3A-5C depict exemplary cores 370, 470, 570. Cores 370, 470, 570 may have any of the features of core 170, discussed above. Each of cores 370, 470, 570 may have a latticed body 372, 472, 572, respectively, and a sheath 374, 474, 574, respectively. Latticed bodies 372, 472, 572 may have any of the properties of latticed body 172, and sheaths 374, 474, 574 may have any of the properties of sheath 174. FIGS. 3A/3B, 4A/4B, and 5A/5B depict only latticed bodies 372, 472, 572, respectively. FIGS. 3A, 4A, and 5A depict cross-sectional views, and FIGS. 3B, 4B, and 5B present perspective views. FIGS. 3C, 4C, and 5C depict cores 370, 470, 570, including sheaths 374, 474, 574, respectively. Cores 370, 470, 570 may be identical except that latticed bodies 372, 472, 572 may have lattice/cell patterns that differ from one another. For example, each of latticed bodies 372, 472, 572 may have a varying lattice/cell pattern selected to balance surface area, cell size, back pressure, and thermal conductivity between coil 162 and the respective latticed bodies 372, 472, 572.

Although latticed bodies 372, 472, 572 are separately described below, it will be appreciated that the properties described below may be combined with one another in any combination. Shapes and structures may be mixed and matched to generate a wide variety of patterns. The patterns of latticed bodies 372, 472, 572, below, are merely exemplary. A wide variety of structures/patterns may be used within the scope of this disclosure.

As shown in FIGS. 3A and 3B, a cross-sectional layer latticed body 372 of core 370 may include struts that are clover/rounded “X” shapes. In an example, this same pattern may be repeated in layers throughout a length of latticed body 372. The layers may be interconnected to one another via struts that extend at least partially along a longitudinal axis of latticed body 372, such that latticed body 372 is an integral piece. As shown in FIGS. 3A and 3B, layers having clover/rounded “X” shapes may be offset/rotated relative to one another. Cross-sectional slices of latticed body 372 may not be uniform and not every cross-section may include clover/rounded “X” shapes. For example, certain cross-sectional slices may only contain struts that connect clover/rounded “X” shaped struts but are not themselves clover/rounded “X” shaped. In one example, alternating layers may be used, where the clover/rounded “X” shaped struts are offset from one another (e.g., so that the clover/rounded “X” shapes are shifted in a direction parallel to the arms of the clover/rounded “X” shapes) between the layers. The layers may repeat in a pattern (e.g., every other layer may be the same) throughout a thickness of latticed body 372. Alternatively, other patterns of layers may be used. Furthermore, the layers may alternatively have different shaped struts from one another.

The spaces between the clover/rounded “X” shaped struts and the struts connecting the various layers may form openings through which fluid may pass. All of the openings may be in fluid communication with one another. Water, or another fluid, may thus be passed from a first end of latticed body 372 to a second end of latticed body 372. The fluid may also pass laterally (perpendicularly to a longitudinal axis of latticed body 372) through latticed body 372. Fluid traveling through latticed body 372 may traverse a variety of paths, contacting the struts of latticed body 372 as it travels through latticed body 372. As discussed above, the struts of latticed body 372 may heat the fluid.

FIGS. 4A-4C depict an alternative latticed body 472. As discussed above with respect to latticed body 372, latticed body 472 may include a plurality of layers that are stacked upon one another along a longitudinal axis of latticed body 472. As shown particularly in the cross-sectional view of FIG. 4A, a layer of latticed body 472 may include struts in an approximately grid-like pattern. The struts may define a plurality of openings, which may be rounded, rounded-squares, or an alternative shape. Struts may extend longitudinally from the grid-like pattern (e.g., midway along a strut defining a side of one of the plurality of openings) to join the plurality of layers together. The struts connecting the layers may leave openings through latticed body 472 in a lateral direction, perpendicular to the longitudinal axis of latticed body 472. The openings of latticed body 472 may all be in fluid communication with one another.

The layers of latticed body 472 may all be the same and have the same orientation, such that the openings are aligned with one another along the longitudinal axis of latticed body 472. Alternatively, the layers of latticed body 472 may be offset from one another such that the openings are not aligned. For example, layers of latticed body 472 may be rotated relative to one another or may be laterally offset from one another. The layers of latticed body 472 (and/or the other latticed bodies described herein) may also be angled such that planes defined by the layers are not normal to the longitudinal axis. The layers may have different angles relative to one another or the same angle.

As discussed above relative to latticed body 372, fluid may flow in a general longitudinal direction through latticed body 472. The fluid may take a variety of paths, traveling longitudinally and laterally as it moves in an overall longitudinal direction from a first end of latticed body 472 to a second end of latticed body 472. The struts of latticed body 472 may be heated, as described above relative to FIG. 2. The heated struts may transmit heat to the fluid, causing it to vaporize.

FIGS. 5A-5C depict an alternative latticed body 572. Latticed body 572 may have an approximately fishnet pattern. Openings of latticed body 572 may be oblong-shaped or fish-eye shaped. Alternatively, the openings may have rounded, square, diamond, triangle, fan, or any other suitable shape. The openings may all have the same shape or may have different shapes from one another.

While latticed bodies 372, 472 may have non-uniform cross-sections (discrete struts may join layers to one another), latticed body 572 may have uniform cross-sections. The pattern shown in FIG. 5A-5C may extend along an entire length of latticed body 572. Latticed body 572 may define a plurality of channels extending longitudinally therethrough. The channels may be discrete (not in fluid communication with one another) or may be in fluid communication. For example, openings may be periodically formed in latticed body between the channels.

As discussed above relative to latticed bodies 372, 472, fluid may flow in a general longitudinal direction through latticed body 572. Where the channels are discrete, fluid may be retained within individual channels. Where the channels are in fluid communication, the fluid may take a variety of paths, traveling longitudinally and laterally as it moves in an overall longitudinal direction from a first end of latticed body 572 to a second end of latticed body 572. The struts of latticed body 572 may be heated, as described above relative to FIG. 2. The heated struts may transmit heat to the fluid, causing it to vaporize.

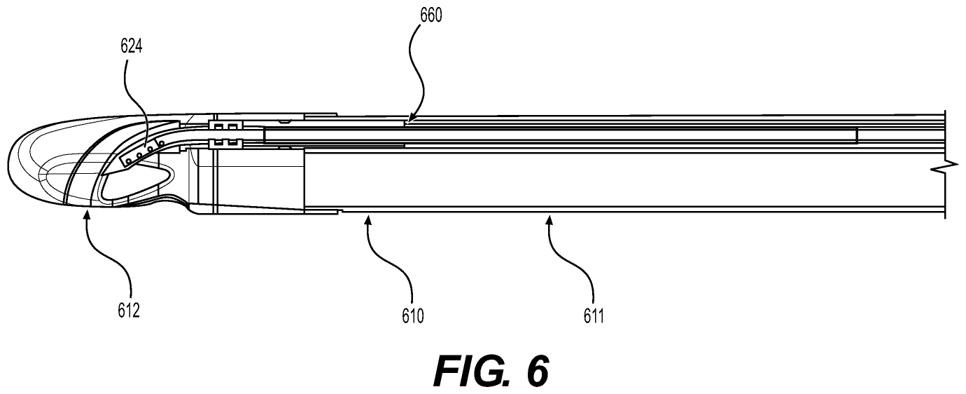

FIG. 6 depicts an exemplary shaft 611 of a vapor ablation device 610. Vapor ablation device 610 may have any of the features of vapor ablation device 10, and shaft 611 may have any of the properties of shaft 11. Shaft 611 may terminate in a distal tip 612 (having any of the properties of distal tip 12). A needle 624 (having any of the properties of needle 24) may extend through a lumen of shaft 611. A vapor generator 660 (having any of the properties of vapor generator 160, described above) may be positioned relative to needle 624. That is, in such an arrangement, needle 624 itself may be formed of Inconel or another similar metal/material and act as a portion of vapor generator 660. In other words, a length of needle 624 may be surrounded (e.g., wrapped) by a portion of vapor generator (e.g., an RF coil similar to RF coil 162). As described above with respect to needle 24, a portion of needle 624 may be extendable and retractable with respect to shaft 611, so that needle 624 may selectively extend outwardly of shaft 611. A distal tip of needle 624 may be bendable to facilitate needle 624 extending radially outward relative to shaft 611.

A portion of needle 624 (e.g., approximately six inches of needle 624) proximal of a bendable portion may include (or form) at least a portion of vapor generator 660, which may be any suitable material (e.g., Inconel). Including vapor generator 660 at least partially within (or as a portion of) needle 624 may provide very pure vapor for delivery to a tissue as such a location minimizes the distance between the vapor generator and the target tissue, thereby minimizing any condensation that may occur along the vapor delivery path. As noted above, vapor delivered from vapor generator 660 may have a small distance to travel, as compared to other locations of a vapor generator. Such positioning of vapor generator 660 within (or as a part of) needle 624 may also reduce or eliminate a need to cool an outer jacket of needle 624. For example, as opposed to arrangements where the vapor generator is positioned elsewhere, such as a handle, thereby requiring temperatures of the handle and the entirety of the shaft to be controlled so as not to exceed an acceptable temperature threshold for handling, the positioning of vapor generator 660 along a distal end of shaft 611 (within or as part of needle 624) permits cooling of only this portion of shaft 611.

In one example, a sheath of needle 624 may surround vapor generator 660. In another example, generator 660 may form an outer surface of needle 624. Vapor generator 660 may include a coil 162, as described above, with respect to FIG. 2. Some portions of generator 660 (e.g., coil 162) may be disposed outside of (e.g., radially surrounding) needle 624, while a core (having any of the properties of cores 170, 370, 470, 570) may be disposed within needle 624.

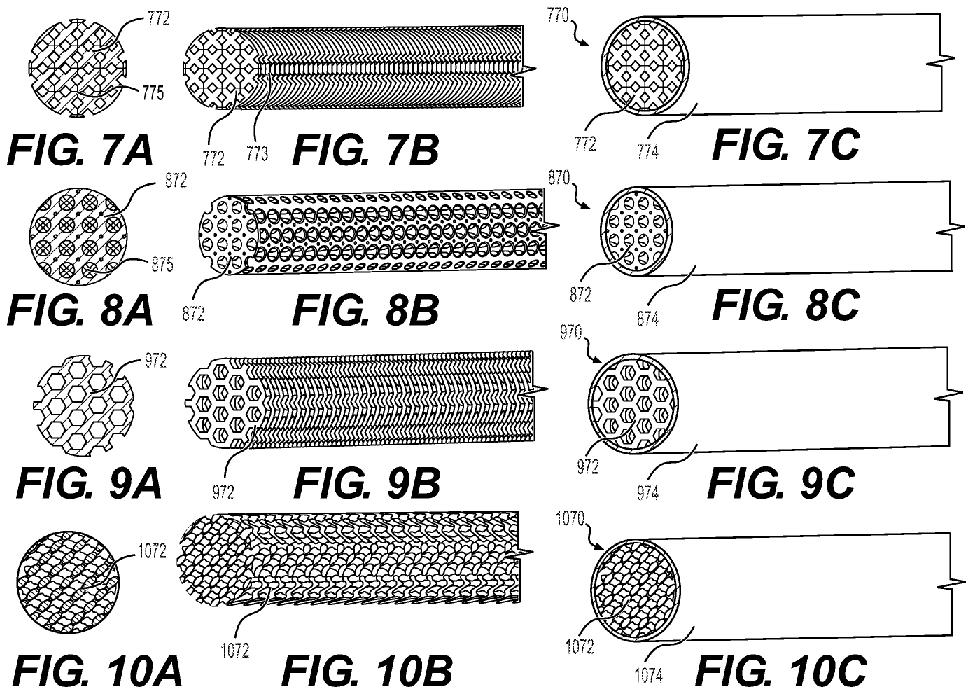

FIGS. 7A-10C depict exemplary cores 770, 870, 970, 1070 for use with vapor generator 660. Exemplary cores 770, 870, 970, 1070 may also be used with vapor generator 160. Cores 770, 870, 970, 1070 may have any of the features of cores 170, 370, 470, 570, discussed above. Each of cores 770, 870, 970, 1070 may have a latticed body 772, 872, 972, 1072, respectively, and a sheath 774, 874, 974, 1074, respectively. Latticed bodies 772, 872, 972, 1072 may have any of the properties of latticed bodies 172, 372, 472, 572, and sheaths 774, 874, 974, 1074 may have any of the properties of sheaths 174, 374, 474, 574. FIGS. 7A/7B, 8A/8B, 9A/9B, and 10A/10B depict only latticed bodies 772, 872, 972, 1072, respectively. FIGS. 7A, 8A, 9A, and 10A depict cross-sectional views, and FIGS. 7B, 8B, 9B, and 10B present perspective views. FIGS. 7C, 8C, 9C, and 10C depict cores 770, 870, 970, 1070, including sheaths 774, 874, 974, 1074, respectively. Cores 770, 870, 970, 1070 may be identical except that latticed bodies 772, 872, 972, 1072 may have lattice/cell patterns that differ from one another. For example, each of latticed bodies 772, 872, 972, 1072 may have a varying lattice/cell pattern selected to balance surface area, cell size, back pressure, and thermal conductivity between coil 162 and the respective latticed bodies 772, 872, 972, 1072. The lattice patterns shown in FIGS. 7A-10C are merely exemplary. Alternative lattice patterns may be used, including those of latticed bodies 372, 472, and 572. The properties of the latticed bodies disclosed herein may be combined in any manner, and features of the latticed bodies may be mixed and matched.

FIGS. 7A-7C depict an exemplary latticed body 772. As discussed above with respect to latticed body 372, 472, and 572, latticed body 772 may include a plurality of layers that are stacked upon one another along a longitudinal axis of latticed body 772. As shown particularly in the cross-sectional view of FIG. 7A, a layer of latticed body 772 may include struts that define square or rectangular-shaped cutouts/openings arranged in rows and columns. The layers may be joined at junctions 773 on an outer surface of latticed body 772. For example, layers may be joined together at 2, 3, 4, or more junctions. Struts 775 may extend between the layers (e.g., midway between vertices of four adjacent square/rectangular openings) to join the plurality of layers together. Alternatively, struts 775 may be omitted. The struts connecting the layers may leave openings through latticed body 772 in a lateral direction, perpendicular to the longitudinal axis of latticed body 772. The openings of latticed body 772 may all be in fluid communication with one another.

The layers of latticed body 772 may all be the same and may be oriented the same, such that the openings are aligned with one another along the longitudinal axis of latticed body 772. Alternatively, the layers of latticed body 772 may be offset from one another such that the openings are not aligned. For example, layers of latticed body 772 may be rotated relative to one another or may be laterally offset from one another. The layers of latticed body 772 (and/or the other latticed bodies described herein) may also be angled such that planes defined by the layers are not normal to the longitudinal axis. The layers may have different angles relative to one another or the same angle.

As discussed above relative to latticed bodies 372, 472, and 572 fluid may flow in a general longitudinal direction through latticed body 772. The fluid may take a variety of paths, traveling longitudinally and laterally as it moves in an overall longitudinal direction from a first end of latticed body 772 to a second end of latticed body 772. The struts of latticed body 772 may be heated, as described above relative to FIG. 2. The heated struts may transmit heat to the fluid, causing it to vaporize.

FIGS. 8A-8C depict an exemplary latticed body 872. As discussed above with respect to latticed bodies 372, 472, 572, and 772 latticed body 872 may include a plurality of layers that are stacked upon one another along a longitudinal axis of latticed body 872. As shown particularly in the cross-sectional view of FIG. 8A, a layer of latticed body 872 may include struts that define round openings/cutouts arranged in rows and columns. Struts 875 may extend between the layers to join the plurality of layers together. The struts 875 connecting the layers may leave openings through latticed body 872 in a lateral direction, perpendicular to the longitudinal axis of latticed body 872. For example, as shown in FIG. 8B, circular openings may be formed to allow lateral passage of water (e.g., latticed body 872 may have circular openings defining planes that are parallel to a longitudinal axis of latticed boy 872). The openings of latticed body 872 may all be in fluid communication with one another.

The layers of latticed body 872 may all be identical and may be oriented the same, such that the openings are aligned with one another along the longitudinal axis of latticed body 872. Alternatively, the layers of latticed body 872 may be offset from one another such that the openings are not aligned. For example, layers of latticed body 872 may be rotated relative to one another or may be laterally offset from one another. The layers of latticed body 872 (and/or the other latticed bodies described herein) may also be angled such that planes defined by the layers are not normal to the longitudinal axis. The layers may have different angles relative to one another or the same angle.

As discussed above relative to latticed bodies 372, 472, 572, 772, fluid may flow in a general longitudinal direction through latticed body 872. The fluid may take a variety of paths, traveling longitudinally and laterally as it moves in an overall longitudinal direction from a first end of latticed body 872 to a second end of latticed body 872. The struts of latticed body 872 may be heated, as described above relative to FIG. 2. The heated struts may transmit heat to the fluid, causing it to vaporize.

FIGS. 9A-9C depict an exemplary latticed body 972. As discussed above with respect to latticed bodies 372, 472, 572, 772, and 872, latticed body 972 may include a plurality of layers that are stacked upon one another along a longitudinal axis of latticed body 972. As shown particularly in the cross-sectional view of FIG. 9A, a layer of latticed body 972 may include struts that define hexagonal openings/cutouts arranged in rows. The struts may have a honeycomb pattern. Struts may extend between struts of the layers to join the plurality of layers together. The longitudinal struts may extend parallel to a longitudinal axis of latticed body 972, between the layers, such that the hexagonal openings may be aligned with one another without obstruction. The struts connecting the layers may leave openings through latticed body 972 in a lateral direction, perpendicular to the longitudinal axis of latticed body 972. The openings of latticed body 972 may all be in fluid communication with one another.

The layers of latticed body 972 may all be the same and may be oriented the same, such that the openings are aligned with one another along the longitudinal axis of latticed body 972. Alternatively, the layers of latticed body 972 may be offset from one another such that the openings are not aligned. For example, layers of latticed body 972 may be rotated relative to one another or may be laterally offset from one another. The layers of latticed body 972 (and/or the other latticed bodies described herein) may also be angled such that planes defined by the layers are not normal to the longitudinal axis. The layers may have different angles relative to one another or the same angle.

As discussed above relative to latticed bodies 372, 472, 572, 772, and 872, fluid may flow in a general longitudinal direction through latticed body 972. The fluid may take a variety of paths, traveling longitudinally and laterally as it moves in an overall longitudinal direction from a first end of latticed body 972 to a second end of latticed body 972. The struts of latticed body 972 may be heated, as described above relative to FIG. 2. The heated struts may transmit heat to the fluid, causing it to vaporize.

FIGS. 10A-10C depict an exemplary latticed body 1072. Latticed body 1072 may have a woven pattern, a knit pattern, or a crocheted pattern. The struts may allow fluid flow longitudinally and laterally through latticed body 1072 and may generate turbulence within the fluid. As shown particularly in the cross-sectional view of FIG. 10A, a layer of latticed body 1072 may include struts that define parallelogram openings/cutouts arranged in rows. The openings of latticed body 1072 may all be in fluid communication with one another, or only a subset of openings may be in fluid communication with one another.

As discussed above relative to latticed bodies 372, 472, 572, 772, 872, and 972, fluid may flow in a general longitudinal direction through latticed body 1072. The fluid may take a variety of paths, traveling longitudinally and laterally as it moves in an overall longitudinal direction from a first end of latticed body 1072 to a second end of latticed body 1072. The struts of latticed body 1072 may be heated, as described above relative to FIG. 2. The heated struts may transmit heat to the fluid, causing it to vaporize.

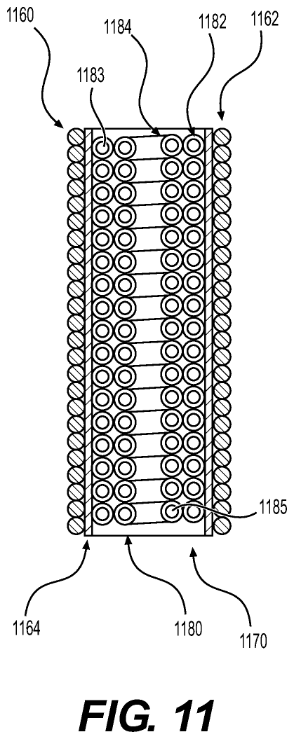

FIG. 11 depicts an alternative vapor generator 1160. Vapor generator may have any of the properties of vapor generators 160 or 660, except as described herein. Vapor generator 1160 may include an RF coil 1162, which may have any of the properties of RF coil 162. Vapor generator 1160 may also have a heating core 1180, which may include one or more coils. In an example, heating core 1170 may include a plurality of fluid coils, including a first fluid coil 1182, and a second fluid coil 1184. As shown, second fluid coil 1184 may be located radially within first fluid coil 1182 such that first and second fluid coils 1182, 1184 are concentrically arranged. Heating core 1180 may include alternative numbers of coils (e.g., three or more coils) which may be concentrically arranged with each other.

Fluid coils 1182, 1184 may define one of more fluid pathways through which fluid from a source of fluid may travel. Fluid coil 1182 may be encapsulated by a sheath 1164 which may insulate fluid coils 1182, 1184 from RF coil 1162, or vice versa. As such, sheath 1164 may be formed of a high temperature thermoplastic such as polyimide. Sheath 1164 may be insulative so as to prevent a direct flow of current from RF coil 1162 to fluid coil(s) 1182 and/or 1184. RF coil 1162 may heat fluid coils 1182, 1184, as described above with respect to RF coil 162 of vapor generator 160.

As noted above, first fluid coil 1182 and second fluid coil 1184 may be concentrically arranged. For example, second fluid coil 1184 may be disposed within first fluid coil 1182. A first lumen 1183 may extend through first fluid coil 1182, and a second lumen 1183 may extend through second fluid coil 1184. First lumen 1183 and second lumen 1185 may be in fluid communication with one another. Fluid may pass through first lumen 1183 and second lumen 1185 during heating. Fluid may first pass through one of fluid coils 1182, 1184, and then through the other of fluid coils 1182, 1184. For example, a fluid source may provide fluid to one end of one of fluid coils 1182, 1184. Fluid may travel through lumen 1183 or 1185 to the other end of the one of fluid coils 1182, 1184. Fluid may then pass into lumen 1183 or 1185 of the other of fluid coils 1182, 1184 and may flow through lumen 1183 or 1185 until it reaches an end of the other of fluid coils 1182, 1184. Lumens 1183 and 1185 may be directly joined together such that fluid enters and exits at the same end of vapor generator 1160. Alternatively, a piece of tubing (not shown) may span lumens 1183 and 1185. In such a configuration, fluid may flow in one end of vapor generator 1160 and out the other end of vapor generator 1160 or may flow into and out of the same end of vapor generator 1160. It is understood that fluid coils 1182, 1184 may be formed via 3D printing such that numerous configurations of coils 1182, 1184 may be realized. For example, in some arrangements, multiple independent fluid pathways may be formed, or one continuous winding pathway may be formed. In some arrangements, a fluid coil may start at one end, wind upwardly in a circular fashion toward a second end, then fold (e.g., invert, turn) in on itself to form an inner coil and wind downwardly back toward the first end. In another arrangement, a first coil layer of a fluid coil may wind in a first direction (e.g., clockwise) to form a circular or semi-circular shaped outer segment, and then turn in towards a center of the segment and wind in a second direction (e.g., counter-clockwise) to form an inner coil segment. This pattern may then be repeated forming numerous layers along a longitudinal length.

Fluid coils 1182 and 1184 may be formed of any suitable material, such as Inconel. Fluid coils 1182 and 1184 may be formed of the same material or from different materials. A material may also vary along a single coil. A thickness of walls of fluid coils 1182, 1184 may be uniform or may be varied in order to provide a desired heating profile. Fluid coils 1182 and/or 1184 may be formed by winding a tubing. Alternatively, fluid coils 1182 and/or 1184 may be formed via additive manufacturing, including any of the techniques described above with respect to vapor generator 160.

Generator 1160 may provide benefits over a generator that uses only one fluid coil. Generator 1160 may provide the same length of fluid travel within a smaller footprint, due to the concentric coils. The smaller footprint may have any of the benefits described above, with respect to generator 160. Indeed, generator 1160 may produce higher quality vapor than previously existing generators as a length of the heating pathway may be doubled or otherwise increased, thereby increasing the amount of time the fluid is positioned inside the heating coil.

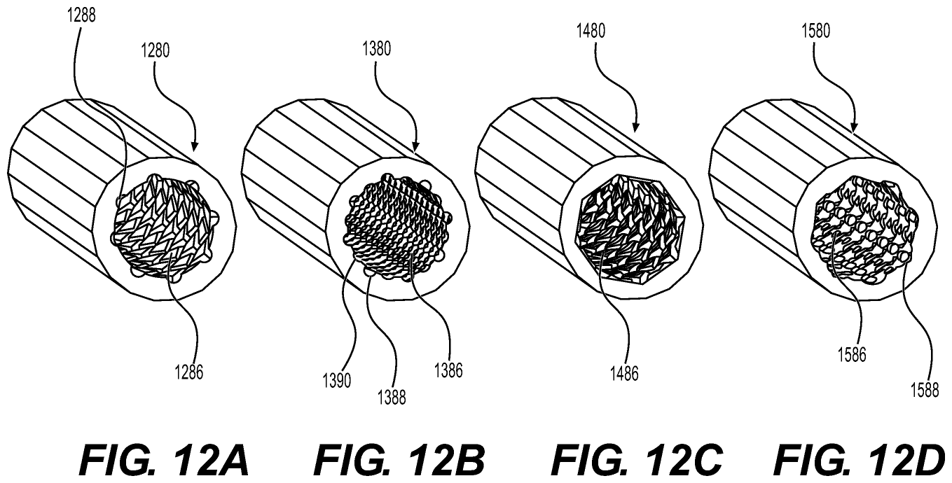

FIGS. 12A-12D depict exemplary tubing having textured inner surfaces. The tubing may be used to form a coil or other structure for use in a generator such as vapor generator 1160, or any other vapor generator. The tubing may define one or more fluid pathways for a flow of fluid from a fluid source to traverse. The textured surfaces of the tubing of FIGS. 12A-12D may provide a higher surface area for efficient transfer of energy in a vapor generator, such as vapor generator 1160. The textured surfaces may increase turbulence of the fluid, further facilitating efficient heating. The tubing of FIGS. 12A-12D may also be used with other vapor generators, including generators that only use a single coil for carrying fluid. The textured surfaces may be selected so as to maximize the surface area thereof while promoting turbulent flow. The tubing depicted in FIGS. 12A-12D may be formed of any suitable material, such as Inconel and by any suitable method, such as any of the additive manufacturing techniques described herein. The tubing may be formed of the same material or from different materials. A material may vary within a single tubing.

FIG. 12A depicts a first tubing 1280. As shown in FIG. 12A, at least one cross-section of tubing 1280 may include a plurality of dimples 1288. Dimples 1288 may be cross-sections of channels, which may extend along an inner surface 1286 of tubing 1280. Dimples 1288 may have an approximately rounded cross-section, because at least some of the channels may have rounded bottoms. Channels may extend annularly about the inner surface 1286 and/or along a longitudinal direction of tubing 1280. The channels may have uniform widths and/or variable widths. The channels may form diamond and/or triangle shapes in inner surface 1286. For example, some of the channels may extend annularly about the inner surface 1286 of tubing 1280, parallel to one another. Other of channels may spiral about inner surface 1286 of tubing 1280, or otherwise extend diagonally along inner surface 1286, thereby forming triangles with the annular channels. A texture of inner surface 1286 may cause turbulence in the fluid, and may provide a greater surface area for contacting the fluid than a smooth inner surface would. The texture of inner surface 1286 may thus facilitate improved heating of the fluid and improved vapor generation.

FIG. 12B depicts an exemplary tubing 1380, having an inner surface 1386. In a cross-section, a wall of tubing 1380 may have larger dimples 1388 and smaller dimples 1390, each of which may have rounded shapes. Larger dimples 1388 may be portions of larger recesses formed in inner surface 1386. Smaller dimples 1390 may be portions of smaller recesses formed in inner surface 1386. The recesses of inner surface 1386 may have varying sizes and profiles. For example, the recesses may form a fish scale type pattern or any suitable type of pattern. A texture of inner surface 1386 may cause turbulence in the fluid, and may provide a greater surface area for contacting the fluid than a smooth inner surface would. The texture of inner surface 1386 may thus facilitate improved heating of the fluid and improved vapor generation.

FIG. 12C depicts an exemplary tubing 1480, having an inner surface 1486. In a cross-section, a wall of tubing 1480 may have flat faces separated by corners, such as the eight flat faces and eight corners depicted in FIG. 12C. The corners may be cross-sections of channels, which may extend along an inner surface 1486 of tubing 1480. Channels may extend in annular directions about the inner surface 1486 and/or along a longitudinal direction of tubing 1480. The channels may have uniform widths and/or variable widths. The channels may form diamond and/or triangle shapes in inner surface 1486. For example, some of the channels may extend annularly about the inner surface 1486 of tubing 1480, parallel to one another. Other of channels may spiral about inner surface 1486 of tubing 1280, or otherwise extend diagonally along inner surface 1486, thereby forming triangles with the annular channels. A texture of inner surface 1486 may cause turbulence in the fluid, and may provide a greater surface area for contacting the fluid than a smooth inner surface would. The texture of inner surface 1486 may thus facilitate improved heating of the fluid and improved vapor generation.

FIG. 12D depicts an exemplary tubing 1580, having an inner surface 1586. In a cross-section, a wall of tubing 1580 may have dimples 1588, each of which may have multiple segments (e.g., rounded or straight segments). Dimples 1588 may be cross-sections of recesses formed in inner surface 1586. The recesses of inner surface 1586 may have varying sizes and profiles, including varying depths and cross-sectional sizes. A texture of inner surface 1586 may cause turbulence in the fluid, and may provide a greater surface area for contacting the fluid than a smooth inner surface would. The texture of inner surface 1586 may thus facilitate improved heating of the fluid and improved vapor generation.

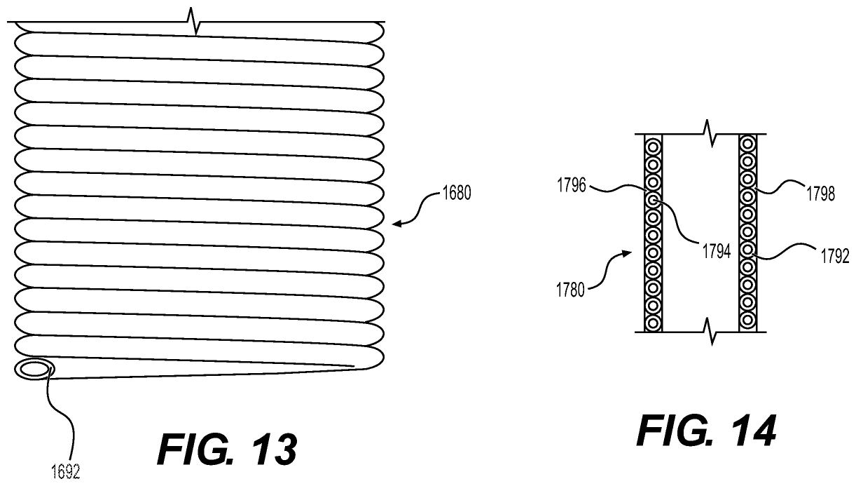

FIG. 13 depicts an exemplary coil 1680, which may be used in a generator such as generator 1160 or any other generator using a coil to carry fluid for heating. Coil 1680 may define a fluid pathway through which fluid from a fluid source may travel. The features of coil 1680 may be used together with features of FIGS. 12A-12D. As shown in FIG. 13, a wall 1692 of coil 1680 may have an elongated shape, thereby defining a lumen having an elongated cross-section. For example, as shown in FIG. 13, the walls may have an oval shape and may define an oval opening. In other examples, the walls may be racetrack shaped, rectangular, ovoid, elliptical, or another suitable shape to define a lumen with an elongated cross-section. The shape of the lumen of coil 1680 may provide for more efficient heating of fluid passing therethrough. A decreased distance from wall 1692 of coil 1680 to a center of the lumen (along a minor axis of a cross-section of coil 1680) may provide for better conduction of heat to the fluid traveling therein. For example, a vapor formed by heating fluid passing therethrough may be less wet when compared to a vapor generated by a coil with a round cross-section. Coil 1680 may be formed of any suitable material, such as Inconel, and by any suitable method, such as any of the additive manufacturing techniques described herein. A material of coil 1680 may be uniform or non-uniform, in order to provide efficient heating. Widths of wall 1692 may be uniform or may vary in order to efficiently heat fluid passing through the lumen of coil 1680.

FIG. 14 depicts an exemplary coil 1780, which may be used in a generator such as generator 1160 or any other generator using a coil to carry fluid for heating. Coil 1780 may define a fluid pathway through which fluid from a fluid source may travel. The features of coil 1780 may be used together with features of FIGS. 12A-12D and 13. As shown in FIG. 13, a wall 1792 of coil 1780 may define a lumen 1794 having any appropriate cross-sectional shape and size. For example, as shown in FIG. 14, wall 1792 may define lumen 1794 with a circular cross-sectional shape. In an ordinary coil having walls with a circular cross-section, approximately triangular-shaped gaps may be formed between the windings. In the coil 1780 of FIG. 14, infill 1796 may fill a gap that would otherwise be present on an inner diameter of coil 1780. Infill 1798 may fill a gap that would otherwise be present on an outer diameter of coil 1780. Only one of infill 1796/1798 may be used, such that only an inner diameter or only an outer diameter has infill. Alternatively, both of infills 1796/1798 may be used. Infills 1796 and 1798 may fill only some of the gaps or may fill all of the gaps. For example, as shown in FIG. 14, infill 1796 may form a smooth, straight inner diameter of coil 1780, and infill 1798 may form a smooth, straight outer diameter of coil 1780. Infill 1796 and/or 1798 may be formed of a uniform material with walls 1792, such that coil 1780 forms a cylinder with helical lumen 1794 formed therein. Separate walls 1792 are shown for illustrative purposes in FIG. 17, but a single, uniform structure may be formed such that circular walls 1792 would not be present.

Eliminating gaps that would otherwise exist with a coil having a rounded cross-section may provide for more efficient heat transfer along coil 1780. Heat may conduct through coil 1780 (and any of the cores, latticed bodies, and coils disclosed herein), and infill 1796, 1798 may improve such heat transfer and thereby provide higher quality vapor and may result in a shorter path of travel required for fluid, thereby allowing a length of coil 1780 to be decreased. A decrease in length of coil 1780 may provide for the space-saving efficiencies described above with respect to generator 160. Coil 1780 may be formed of any suitable material, such as Inconel and by any suitable method, such as any of the additive manufacturing techniques described herein. A material of coil 1780 may be uniform or non-uniform, in order to provide efficient heating. Widths of wall 1792 and/or infill 1796, 1798 may be uniform or may vary in order to efficiently heat fluid passing through the lumen of coil 1780.

Any of the devices described herein may have additional features to provide for more efficient heat and/or energy transfer. For example, the examples described herein may include features that are formed by any of the additive manufacturing methods described herein. For example, features such as thickened or flattened portions of any of the coils or cores disclosed herein may be formed to provide for improved connections between thermocouples and/or Litz wires and the coils/cores. The thickened or flattened portions may provide landing pads for the wires of thermocouples, Litz wires, or other structures, so as to improve welding of various structures by adding robust areas/thickened portions to which the thermocouples, Litz wires, or other structures adhere so as to avoid or reduce areas/welds susceptible to breaking or leaking.

While principles of the present disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A vapor generator for use in a medical device, the vapor generator including:
a heating core in fluid communication with a source of fluid and defining at least one fluid pathway along which fluid from the source of fluid travels, wherein the at least one fluid pathway includes one or more surfaces that generate turbulence in the fluid; and
a coil disposed about the heating core, wherein the coil is configured to receive a current so as to heat the fluid traveling along the at least one fluid pathway, thereby generating a vapor.

2. The vapor generator of claim 1, wherein the coil is configured to inductively heat the fluid.

3. The vapor generator of claim 1, wherein the heating core includes a latticed body, and wherein one or more struts of the latticed body define the one or more surfaces that generate the turbulence in the fluid.

4. The vapor generator of claim 3, wherein the heating core further includes a sheath disposed about the latticed body.

5. The vapor generator of claim 3, wherein the latticed body includes Inconel.

6. The vapor generator of claim 3, wherein the latticed body has an approximately cylindrical shape.

7. The vapor generator of claim 3, wherein the latticed body defines a plurality of openings defined by a plurality of struts.

8. The vapor generator of claim 7, wherein all of the openings are in fluid communication with one another.

9. The vapor generator of claim 3, wherein the at least one fluid pathway defines a plurality of routes of fluid travel along (a) a path approximately parallel to a longitudinal axis of the latticed body and (b) a path transverse to the longitudinal axis of the latticed body.

10. The vapor generator of claim 1, wherein at least a portion of the heating core is disposed within a needle of the medical device.

11. The vapor generator of claim 10, wherein a portion of the needle having the heating core is disposed within a shaft insertable into a body lumen of a subject.

12. The vapor generator of claim 1, wherein the heating core includes a tube, and wherein the tube defines a lumen having a textured wall surface, wherein the textured wall surface generates the turbulence in the fluid.

13. The vapor generator of claim 12, wherein the tube forms a coil.

14. The vapor generator of claim 13, wherein the heating core includes two or more coils.

15. The vapor generator of claim 12, wherein the tube has a non-circular cross-section.

16. A vapor generator for use in a medical device, the vapor generator including:
a latticed body in fluid communication with a source of fluid and defining a plurality of fluid pathways along which fluid from the source of fluid travels, wherein the latticed body includes a plurality of struts defining a plurality of openings; and
a coil disposed about the latticed body, wherein the coil is configured to receive a current so as to heat the fluid traveling along the plurality of fluid pathways, thereby generating a vapor.

17. The vapor generator of claim 16, wherein the plurality of fluid pathways define routes of fluid travel along (a) a path approximately parallel to a longitudinal axis of the latticed body and (b) a path transverse to the longitudinal axis of the latticed body.

18. The vapor generator of claim 16, wherein at least a portion of the latticed body is disposed within a needle of the medical device.

19. The vapor generator of claim 16, wherein the latticed body has an approximately cylindrical shape.

20. A vapor generator for use in a medical device, the vapor generator including:
a latticed body in fluid communication with a source of fluid and defining a plurality of fluid pathway along which fluid from the source of fluid travels, wherein the plurality of fluid pathways define routes of fluid travel along (a) a path approximately parallel to a longitudinal axis of the latticed body and (b) a path transverse to the longitudinal axis of the latticed body; and
a coil disposed about the latticed body, wherein the coil is configured to receive a current so as to heat the fluid traveling along the plurality of fluid pathways, thereby generating a vapor.
